(19) United States
(12) Patent Application Publication
(10) Pub. No.: US 2026/0344684 A1
(43) Pub. Date: Nov. 5, 2026

(54) Baking Method For Pulse Protein Fortified Bread Employing Thermostable Amyglucosidase Variant (EC 3.2.1.3)

(71) Applicant: Novozymes A/S, Bagsvaerd (DK)

(72) Inventors: Henrik LUNDKVIST, Bagsvaerd (DK); Camilla VARMING, Bagsvaerd (DK)

(73) Assignee: Novozymes A/S, Bagsvaerd (DK)

(21) Appl. No.: 19/119,928

(22) PCT Filed: Nov. 30, 2022
(86) PCT No.: PCT/EP2022/083877

§ 371 Date: Apr. 10, 2025

(30) Foreign Application Priority Data
Oct. 24, 2022 (EP) 22203319.3

(51) Int. Cl. A21D 8/04; A21D 2/26; A21D 13/064; C12N 9/18; C12N 9/34

(52) U.S. Cl. CPC A21D 8/042; A21D 2/266; A21D 13/064; C12N 9/18; C12N 9/2428; C12Y 302/01003

(57) ABSTRACT

Methods of producing a baked or partially baked product comprising providing a dough comprising added pulse and/or legume protein and a mature thermostable variant of a parent glucoamylase at least 70% identical to SEQ ID NO:1, SEQ ID NO:6, SEQ ID NO:7, SEQ ID NO:8 or SEQ ID NO:10; and baking or partially baking the dough to produce the baked or partially baked product.

### REFERENCE TO SEQUENCE LISTING

This application contains a Sequence Listing in computer readable form, which is incorporated herein by reference.

### FIELD OF THE INVENTION

The invention relates to methods of producing a baked or partially baked product comprising providing a dough comprising added pulse and/or legume protein and a mature thermostable variant of a parent glucoamylase at least 70% identical to SEQ ID NO:1, SEQ ID NO:6, SEQ ID NO:7, SEQ ID NO:8 or SEQ ID NO:10; and baking or partially baking the dough to produce the baked or partially baked product.

### BACKGROUND OF THE INVENTION

Commercial interest in food having a high protein and/or fiber content is increasing due to a growing interest worldwide in healthy food, and high-protein baked products is no exception. However, simply adding flour from a crop that is high in protein, such as pulse flour, i.e. flour made from the edible seeds of plants in the legume family, to a dough has a dramatic and detrimental effect on the volume of the resulting baked product, as shown herein. A technical solution to counter the negative effect on volume is highly desirable.

Par-baking is a technique where a bread or another dough product is partially (i.e. “par”) baked and then typically cooled or frozen for storage. When the final baked product is wanted, the cooled or frozen par-baked product is baked at normal baking temperatures for typically 5 to 15 minutes; the resulting type of baked product is frequently referred to as “bake-off”.

WO 2021/239267 discloses methods of producing baked or partially baked edible products from dough comprising added pulse and/or legume protein and at least one added lipase enzyme, wherein at least 2% (w/w) of the total flour content is added pulse and/or legume protein.

### SUMMARY OF THE INVENTION

The inventors found that thermostable variants of certain glucoamylases showed improved performance in baking with added pulse and/or legume protein. One improved performance of the thermostable variants was that they increased the sweetness or sweet taste of the product, which allowed a reduction in the amount of added sugar in traditional recipes and also to some extent was able to mask otherwise bitter flavours from the added pulse and/or legume protein.

Accordingly in a first aspect, the invention relates to methods of producing a baked or partially baked product comprising providing a dough comprising added pulse and/or legume protein and a mature thermostable variant of a parent glucoamylase at least 70% identical to SEQ ID NO: 1, SEQ ID NO:6, SEQ ID NO:7, SEQ ID NO: 8 or SEQ ID NO: 10; and baking or partially baking the dough to produce the baked or partially baked product.

Preferably, the mature thermostable variant of a parent glucoamylase of the invention is at least 71% identical to SEQ ID NO:1, SEQ ID NO:6, SEQ ID NO:7, SEQ ID NO:8 or SEQ ID NO: 10, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO: 1, SEQ ID NO:6, SEQ ID NO:7, SEQ ID NO:8 or SEQ ID NO: 10.

### FIGURES

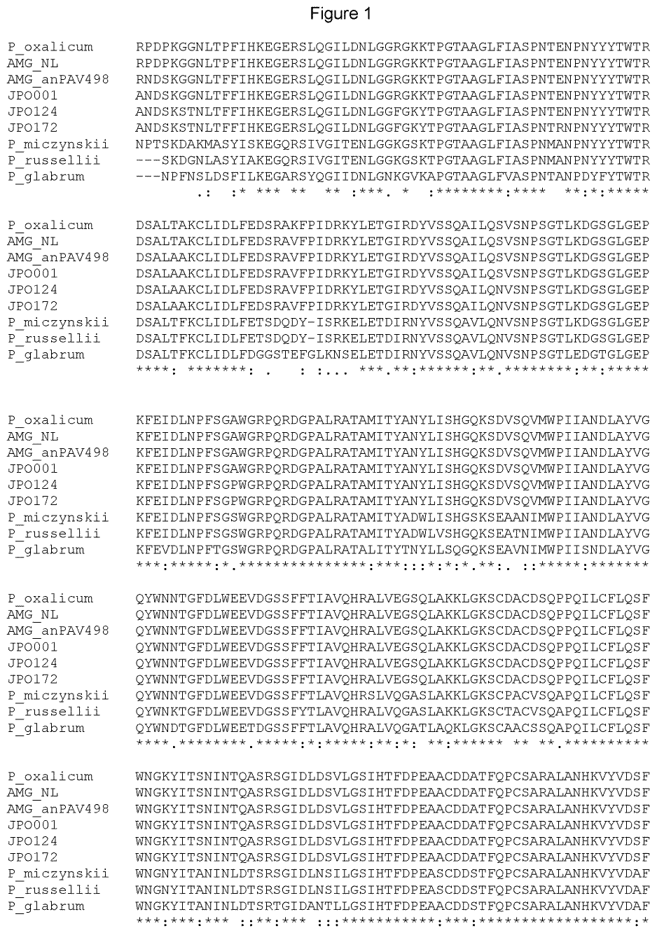

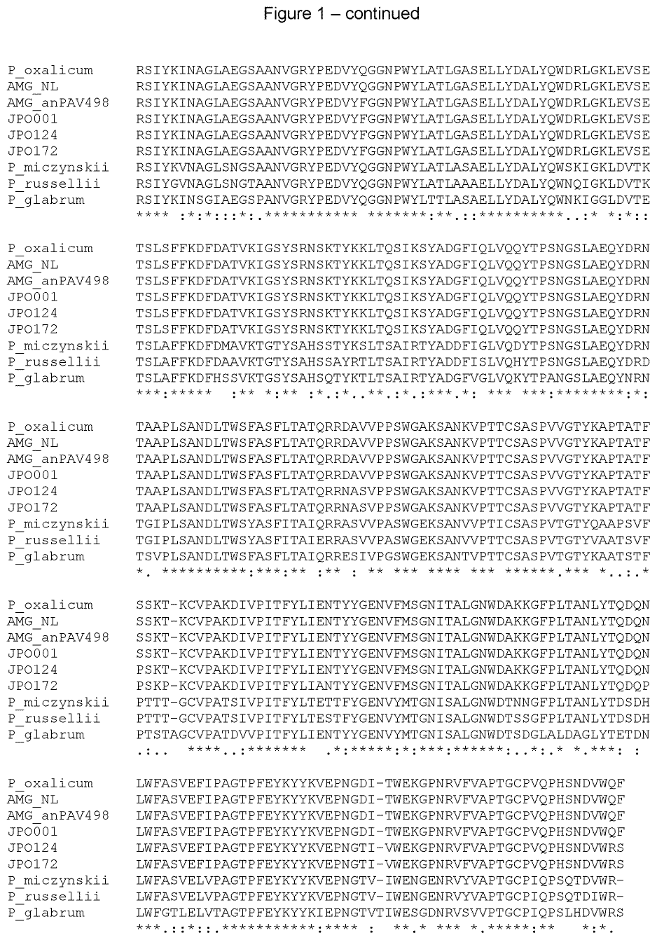

FIG. 1 shows a multiple alignment of the amino acid sequences of the mature proteins of:
Wildtype AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO172’ of SEQ ID NO:6
Wildtype AMG from Penicillium miczynskii (PoAMG) of SEQ ID NO:7
Wildtype AMG from Penicillium russellii (PoAMG) of SEQ ID NO:8
Wildtype AMG from Penicillium glabrum (PoAMG) of SEQ ID NO:9

### DETAILED DESCRIPTION OF THE INVENTION

### Definitions

Legume: Legumes are plants in the Fabaceae family (or Leguminosae), or the fruit or seed of such a plant (also called a pulse, especially in the mature, dry condition). Well-known legumes include alfalfa, clover, beans, peas, chickpeas, lentils, lupins, mesquite, carob, soybeans, peanuts, and tamarind. Legumes produce a botanically unique type of fruit—a simple dry fruit that develops from a simple carpel and usually dehisces (opens along a seam) on two sides.

Pulse: The United Nations Food and Agriculture Organization (FAO) recognizes 11 types of pulses: dry beans, dry broad beans, dry peas, chickpeas, cow peas, pigeon peas, lentils, Bambara beans, vetches, lupins and pulses NES (i.e. minor pulses, including: Lablab, hyacinth bean (Lablab purpureus), Jack bean (Canavalia ensiformis), sword bean (Canavalia gladiata), Winged bean (Psophocarpus tetragonolobus), Velvet bean, cowitch (Mucuna pruriens var. utilis), Yam bean (Pachyrhizus erosus).

Pulse and/or legume protein: The term ‘pulse and/or legume protein’ means pulse protein and/or legume protein, a desirable constituent of pulse flour and/or legume flour; the term also includes processed and/or deflavoured pulse and/or legume flour, wherein the processed flour has a higher protein content than unprocessed flour. Processed or deflavoured pulse and/or legume flour may also be termed pulse and/or legume protein concentrate and/or isolate, respectively.

Deflavoured pulse and/or legume flour or protein: In the context of the instant invention, the term ‘deflavoured’ means that the flour or protein component has been processed to reduce off-flavour, e.g., bitterness.

Lipase activity: Triacylglycerol lipase activity (EC 3.1.1.3), i.e., hydrolytic activity for carboxylic ester bonds in triglycerides, e.g., tributyrin.

Phospholipase activity: Phospholipase activity (A1 or A2, EC 3.1.1.32 or 3.1.1.4), i.e., hydrolytic activity towards one or both carboxylic ester bonds in phospholipids such as lecithin.

Galactolipase activity: Galactolipase activity (EC 3.1.1.26), i.e., hydrolytic activity on carboxylic ester bonds in galactolipids such as DGDG (digalactosyl diglyceride).

Mature polypeptide: The term “mature polypeptide” means a polypeptide in its final form following translation and any post-translational modifications, such as N-terminal processing, C-terminal truncation, glycosylation, phosphorylation, etc.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the -no brief option) is used as the percent identity and is calculated as follows:

(Identical Residues×100)/(Length of Alignment−Total Number of Gaps in Alignment)

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Thermostability improvement: The thermostability improvement (Td) in °C is a measure of how much the variants have improved in thermostability over their parent glucoamylase under the same conditions, determined as exemplified herein.

Improved crumb firmness of the baked product: The term “improved crumb firmness” is defined herein as the property of a baked product that is more easily compressed compared to a baked product wherein the enzyme solution according to the invention is not added to the dough.

The crumb firmness is evaluated either empirically by the skilled test baker/sensory panel or measured by the use of a texture analyzer (e.g., TAXT2 or TA-XT Plus from Stable Micro Systems Ltd, Surrey, UK) as known in the art.

Improved flavor of the baked product: The term “improved flavor of the baked product” is evaluated by a trained test panel and/or chemical analysis (e.g., headspace GC-MS analysis). Improved flavor of the baked product comprises the reduction of off-flavor(s) of the baked product.

Improved anti-staling of the baked product: The term “improved anti-staling of the baked product” is defined herein as the properties of a baked product that have a reduced rate of deterioration of quality parameters, e.g., softness and/or elasticity, during storage.

Volume of the baked product: The term “volume of the baked product” is defined herein as the measure of the volume of a given loaf of bread. The volume may be determined by the rape seed displacement method.

Bread colour: The colour or whiteness of a baked or par-baked product is measured as the “Colour L*” value in a C-cell (Calibre Instruments Ltd, Warrington, UK) using the standard method for collecting images and the standard C-Cell software for data analysis.

### Dough According to the Invention

The invention relates to doughs for baked- or par-baked products, said doughs comprising added pulse and/or legume protein.

The term “added” is defined herein as adding the proteins and/or enzymes according to the invention to the dough, to any ingredient from which the dough is to be made, and/or to any mixture of dough ingredients from which the dough is to be made.

In other words, the proteins and/or enzymes may be added in any step of the dough preparation and may be added in one, two or more steps. They may be added to the ingredients of dough that may be kneaded and processed as known in the art for baked and/or par-baked products.

The term “effective amount” is defined herein as an amount of an enzyme composition according to the invention that is sufficient for providing a measurable effect on at least one property of interest of the dough and/or baked product.

The term “dough” is defined herein as a mixture of flour and other baking ingredients firm enough to knead or roll. In the context of the present invention, batters are encompassed in the term “dough”; preferably the dough of the instant invention comprises wheat flour.

In a preferred embodiment, the dough ingredients comprise wheat flour; preferably 2% (w/w) or more of the total flour content is wheat flour; preferably 4% (w/w) or more of the total flour content is wheat flour, preferably at least 6%, at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or preferably at least 95% (w/w) of the flour is wheat flour.

The dough of the invention may comprise flour derived from any cereal grain or other sources, including wheat, emmer, spelt, einkorn, barley, rye, oat, corn, sorghum, rice, millet, amaranth, quinoa, cassava, and any combination thereof.

In a preferred embodiment of the invention, the pulse and/or legume protein is added to the dough in the form of pulse and/or legume flour, processed pulse and/or legume flour, deflavoured pulse and/or legume flour, or protein concentrate and/or isolate made essentially from pulse and/or legume flour; preferably the added pulse and/or legume protein comprises lentil protein, chickpea protein, pea protein and/or faba bean protein, or a protein concentrate and/or isolate thereof.

A preferred embodiment relates to the dough according to the first aspect, wherein at least 4% (w/w) of the total flour content is added pulse and/or legume protein, preferably at least 6% (w/w) of the total flour content is added pulse and/or legume protein, more preferably at least 8% (w/w) of the total flour content is added pulse and/or legume protein, even more preferably at least 10% (w/w) of the total flour content is added pulse and/or legume protein, most preferably, at least 12% (w/w) of the total flour content is added pulse and/or legume protein

Preferably the dough of the invention also comprises gluten.

The dough may also comprise other conventional dough ingredients, e.g., proteins, such as milk powder, gluten, source of dietary fiber (such as wheat, oat bran, beta-glucan and/or inulin), and eggs (either whole eggs, egg yolks, or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate, or calcium sulfate, and/or an emulsifier.

In a preferred embodiment of the invention, the dough of the invention also comprises gluten.

The dough may comprise fat (triglyceride) such as granulated fat or oil.

The dough of the invention is normally a leavened dough or a dough to be subjected to leavening.

The dough may be leavened in various ways, such as by adding chemical leavening agents, e.g., baking powder, sodium bicarbonate, or by adding a leaven (fermenting dough), but it is preferred to leaven the dough by adding a suitable yeast culture, such as a culture of Saccharomyces cerevisiae (baker's yeast), e.g., a commercially available strain of S. cerevisiae.

The doughs of the invention may comprise at least one added lipase enzyme, preferably a lipase and/or a phospholipase, preferably a mature lipase and/or mature phospholipase. Preferably, the at least one added lipase enzyme comprises a mature lipase having amino acid sequence at least 70% identical to one or more of the sequences shown in SEQ ID NO: 17, SEQ ID NO: 18, SEQ ID NO: 19, SEQ ID NO: 20 and SEQ ID NO: 21; preferably at least 75% identical, at least 80%, 85%, 90%, 92%, 94%, 96%, 98% or preferably at least 99% identical to one or more of the sequences shown in SEQ ID NO: 17, SEQ ID NO: 18, SEQ ID NO: 19, SEQ ID NO: 20 and SEQ ID NO: 21.

Preferably the at least one added lipase enzyme is added in an amount in the range of 0 to 100 mg enzyme protein/kg flour; preferably in the range of 0 to 50 mg enzyme protein/kg flour; more preferably in an amount in the range of 0 to 25 mg enzyme protein/kg flour; even more preferably in an amount in the range of 0 to 10 mg enzyme protein/kg flour; still more preferably in an amount in the range of 0 to 5 mg enzyme protein/kg flour; and most preferably in an amount in the range of 0 to 2.5 mg enzyme protein/kg flour.

Preferably the dough of the first aspect also comprises at least one additional added enzyme, preferably at least one mature alpha-amylase, more preferably a mature maltogenic alpha-amylase; preferably a mature maltogenic alpha-amylase from Bacillus stearothermophilus; more preferably a mature maltogenic alpha-amylase having an amino acid sequence at least 70% identical to that of SEQ ID NO:6, preferably at least 75% identical, at least 80%, 85%, 90%, 92%, 94%, 96%, 98% or preferably at least 99% identical to that of SEQ ID NO: 11. It Is preferred that the mature maltogenic alpha-amylase is added in an amount in the range of 0 to 10.000 MANU/kg flour; preferably in the range of 0 to 7.500 MANU/kg flour; preferably in the range of 0 to 5.000 MANU/kg flour.

Preferably the at least one additional added enzyme comprises a mature alpha amylase; preferably a mature fungal alpha amylase; more preferably a mature alpha amylase from Aspergillus oryzae; preferably the additional mature alpha-amylase is added in an amount in the range of 0 to 1.000 FAU/kg flour; preferably in the range of 0 to 500 FAU/kg flour; more preferably in the range of 0 to 100 FAU/kg flour; even more preferably in the range of 0 to 50 FAU/kg flour; and most preferably in the range of 0 to 25 FAU/kg flour.

Preferably the dough of the first aspect also comprises at least one additional added enzyme comprises at least one mature xylanase, preferably a GH5, a GH8 and/or a GH11 xylanase.

The present invention is particularly useful for preparing yeast-raised dough, baked or par-baked products in industrialized processes in which the dough used to prepare the baked or par-baked products are prepared mechanically using automated or semi-automated equipment. The process of preparing bread generally involves the sequential steps of dough making (with an optional proofing step), sheeting or dividing, shaping or rolling, and proofing, the dough, which steps are well known in the art. If the optional proofing step is used, preferably more flour is added and alkali may be added to neutralize acid produced or to be produced during the second proofing step. In an industrial baked production process according to the present invention, one or more of these steps is/are performed using automated or semi-automated equipment, such as:

Horizontal mixers: Roller bar mixers equipped with rotating arms, which in old models have two speed settings, typically, slow mixing at 35 rpm and fast mixing at 70 rpm, while newer models more often have variable speed settings ranging from 15-120 rpm.

Vertical mixers: Spiral mixers are typically mixers with a rotating bowl and a spiral counteracting the rotation. Some spiral mixers can be bidirectional to provide better distribution of the ingredients.

The purpose of mixing is uniform blending and hydration of dry material, kneading of the dough to form a gluten network and incorporation of air into the dough. Two-speed mixing is usually employed with both types of mixers: A slow speed to collect the dough without pushing the dough to the side of the bowl, and a fast speed to assist formation of the gluten network.

In a preferred embodiment, the dough is mixed:
a) at least 5 minutes at a slow mixing speed, preferably in the range of 5-50 rpm, more preferably in the range of 10-40 rpm; more preferably at least 10 minutes at a slow mixing speed, even more preferably at least 15 minutes a slow mixing speed; and optionally
b) the dough is subsequently mixed at a faster speed.

### Par-Baked Products

Par-baked is a technique in which a bread or a dough product is partially baked and then typically rapidly cooled/frozen for storage.

The raw dough is baked normally, but halted at about approximately 80% of the normal cooking time, where after it is rapidly cooled.

A par-baked dough product bread can be transported easily, and stored until needed. Par-baked dough products are kept in sealed containers that prevent moisture loss. They are may be stored at room temperature; or stored in a fridge, or stored in a freezer.

The freezing step may lead to ice crystal formation and subsequent damage to the starch granules and amylose leakage. It is therefore likely that the amount of leaked amylose and unbound water is higher prior to the second bake-off than in a bread baked without a freezing step. These are two parameters known to increase the crumb firming rate.

When the final dough product is desired, a par-baked product is “finished off” by baking it at normal temperatures for an additional time, typically 5 to 15 minutes. The exact time must be determined by testing, as the time varies depending on the product.

Accordingly, the par-baked product is manufactured by the following steps:
a) the dough is made into a product,
b) the product is baked,
c) the product is stored, and
d) the product is re-baked to a par-baked product.

The product may be stored at ambient/room temperature, or the product may be stored a low temperature, which means that it will normally be stored at a temperature below 5 degrees Celsius. In one embodiment, the product will be stored in a freezer.

The process of the invention may be used for any kind of par-baked product prepared from dough, in particular of a soft character, either of a white, light or dark type.

Examples are bread (in particular white, whole-meal or rye bread), typically in the form of loaves or rolls, bread, flat bread, pita bread, tortillas, cakes, pancakes, biscuits, wafers, cookies, pie crusts, pizza, and the like.

### Glucoamylases

Glucoamylases are also called amyloglucosidases, and Glucan 1,4-alpha-glucosidase (EC 3.2.1.3), more commonly they are referred to as AMGs.

According to the present invention, different types of amyloglucosidases may be used as parent for the generation of a thermostable amyloglucosidase variant, e.g, the amyloglucosidase may be a polypeptide that is encoded by a DNA sequence that is found in a fungal strain of Aspergillus, Rhizopusor, Talaromyces (Rasamsonia) or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Preferably, the parent glucoamylase is from a species of Penicillium, preferably from Penicillium oxicalum, Penicillium miczynskii, Penicillium russellii or Penicillium glabrum.

Examples of other suitable fungi include Aspergillus niger, Aspergillus awamori, Aspergillus oryzae, Rhizopus delemar, Rhizopus niveus, Rhizopus oryzae and Talaromyces emersonii (Rasamsonia emersonii).

Below is shown the %-identity between the AMG amino acid sequences aligned in FIG. 1, and also provided in the sequence list:

| | | | | | | | | | |
|---|---|---|---|---|---|---|---|---|---|
| P_oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO172 | 95.97 | 96.13 | 96.97 | 97.14 | 99.33 | 100.00 | 76.73 | 76.78 | 73.99 |
| P_miczynskii | 77.07 | 77.07 | 76.73 | 76.73 | 77.07 | 76.73 | 100.00 | 94.75 | 80.51 |
| P_russellii | 77.12 | 77.12 | 76.95 | 76.95 | 77.12 | 76.78 | 94.75 | 100.00 | 79.66 |
| P_glabrum | 74.32 | 74.32 | 73.82 | 73.82 | 74.32 | 73.99 | 80.51 | 79.66 | 100.00 |

Thermostable variants of the PoAMG have been generated (see table 2 below). In a preferred embodiment, the mature thermostable glucoamylase variant of the invention comprises one or more or all of the combinations of amino acid substitutions listed in table 2 below.

In a preferred embodiment, the mature variant of the invention comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO:1; preferably the at least one amino acid modification comprises a substitution in one or more or all of the positions corresponding to positions 1, 2, 4, 11, 65, 79 and 327 in SEQ ID NO: 1, preferably the at least one amino acid modification comprises a substitution in one or more or all of the positions corresponding to R1A, P2N, P4S, P11F, T65A, K79V and Q327F in SEQ ID NO:1; or preferably the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO: 1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

The thermostability improvements (Td) of the variants in table 2 are listed in Table 3, where the Td of the PoAMG variant denoted “anPAV498” (the parent) was set to zero. In a preferred embodiment, the the mature thermostable variant of the invention has a thermostability improvement (Td) over its parent of at least 5 °C, preferably at least 6 °C, 7 °C or 8 °C, preferably determined as exemplified herein.

In another preferred embodiment, the mature thermostable variant of the invention has a relative activity at 91 °C of at least 150, preferably at least 200, more preferably at least 250, most preferably at least 300 compared to its parent.

### Sources of Phospholipase Enzymes

The phospholipase enzymes may be prokaryotic, particularly bacterial, or eukaryotic, e.g., from fungal or animal sources.

Phospholipase enzymes may be derived, e.g., from the following genera or species: Thermomyces, T. lanuginosus (also known as Humicola lanuginosa); Humicola, H. insolens; Fusarium, F. oxysporum, F. solani, F. heterosporum; Aspergillus, A. tubigensis, A. niger, A. oryzae; Rhizomucor; Candida, C. antarctica, C. rugosa, Penicillium, P. camembertii; Rhizopus, Rhizopus oryzae; Absidia, Dictyostelium, Mucor, Neurospora, Rhizopus, R. arrhizus, R. japonicus, Sclerotinia, Trichophyton, Whetzelinia, Bacillus, Citrobacter, Enterobacter, Edwardsiella, Erwinia, Escherichia, E. coli, Klebsiella, Proteus, Providencia, Salmonella, Serratia, Shigella, Streptomyces, Yersinia, Pseudomonas, or P. cepacia.

The phospholipase enzyme may be produced in a suitable host cell as known in the art.

Phospholipase may also be obtained from bee or snake venom or from mammal pancreas, e.g., porcine pancreas.

WO 98/26057 discloses a lipase/phospholipase from Fusarium oxysporum and its use in baking.

WO 2004/099400 discloses various phospholipase enzymes and their use in baking for reduction of dough stickiness.

Suitable commercial phospholipase preparations are Lipopan F™, Lipopan Xtra™, and Lipopan Prime™ (available from Novozymes A/S).

Other available phospholipases are, e.g., Panamore™ available from DSM.

Commercial lipase preparations are, e.g., Lipopan FTM and Lipopan 50 BG™ available from Novozymes A/S.

### Alpha-Amylases

Alpha-Amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

A number of alpha-amylases are referred to as Termamyl™, Termamyl® SC and “Termamyl™-like alpha-amylases” and are known from, e.g., WO 90/11352, WO 95/10603, WO 95/26397, WO 96/23873 and WO 96/23874.

Another group of alpha-amylases are referred to as Fungamyl™ and “Fungamyl™-like alpha-amylases”, which are alpha-amylases related to the alpha-amylase derived from Aspergillus oryzae disclosed in WO 01/34784.

A preferred group of alpha-amylases are referred to as maltogenic alpha-amylases (EC 3.2.1.133), typically derived from Bacillus stearothermophilus. Preferred maltogenic alpha-amylases have an amino acid sequence at least 70% identical to SEQ ID NO:11 herein, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO: 11 herein.

Also preferred is the group of sugar-tolerant maltogenic alpha-amylase variants, such as the ones disclosed in WO 2006/032281 (Novozymes A/S), where a number of sugar-tolerant variants are provided, the variants each comprise an amino acid alteration which is a substitution or deletion of or insertion adjacent to 115, R18, K44, N86, T87, G88, Y89, H90, Y92, W93, F188, T189, 0190, P191, A 192, F194, L196, 0329, N371, 0372, P373, N375 or R376. One such preferred variant we denote sugar-tolerant maltogenic alpha-amylase 1 (ST-MAA1) herein and it has the following three substitutions: F188L, D261G and T288P. WO 2008/148845 also discloses a number of preferred sugar-tolerant maltogenic alpha-amylase variants, the variants each comprise the two substitutions D261G and T288P and at least one additional amino acid alteration which is a substitution or deletion of or insertion adjacent to Y89, W93, P191, F194, Y360 or N375. One such preferred variant we denote sugar-tolerant maltogenic alpha-amylase 2 (ST-MAA2) and it has the following four substitutions: F194Y, D261G, T288P and N375S.

Yet another group of preferred mature alpha-amylases are the mature non-maltogenic maltotetrahyrolase exoamylases. WO 2004/111217 (Danisco A/S) discloses a number of preferred non-maltogenic thermostable variants of a Pseudomonas saccharophilia exoamylase or maltotetrahydrolase having the amino acid sequence shown in SEQ ID NO: 12, the variants each comprise one or more of the following substitutions: G69P, A141P, G223A, A268P, G313P, S399P and G400P.

WO 2007/148224 (Danisco A/S) discloses more preferred mature non-maltogenic anti-staling variants of a Pseudomonas saccharophilia exoamylase having the amino acid sequence shown in SEQ ID NO: 12, the variants each comprise an amino acid substitution at position 307 to lysine (K) or arginine (R).

WO 2010/133644 discloses other preferred mature non-maltogenic variants of a Pseudomonas saccharophilia exoamylase having the amino acid sequence shown in SEQ ID NO: 12, the variants each comprise one or more substitutions in positions including position 42, 88, 205, 223, 235, 240, 311, 392, and 409. One such preferred variant of the Pseudomonas saccharophilia exoamylase is disclosed in SEQ ID NO:31 of WO 2010/133644, its amino acid sequence is also shown in SEQ ID NO: 13 herein and we denote it: HPL G+.

Another preferred variant of the Pseudomonas saccharophilia exoamylase is disclosed in SEQ ID NO:21 of WO 2007/148224, its amino acid sequence is also shown in SEQ ID NO: 14 herein and we denote it: HPL G4.

Other preferred non-maltogenic alpha-amylases are disclosed in WO2005003339 (Danisco A/S), WO2005007818 (Danisco A/S) and WO 2022/216801 (DuPont Nutrition Biosciences ApS).

A preferred mature non-maltogenic alpha-amylase has an amino acid sequence at least 70% identical to SEQ ID NO: 12, 13 or 14 herein, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO: 12, 13 or 14 herein; in addition the preferred mature non-maltogenic alpha-amylase has one or more of the substitutions listed in the above paragraph.

Still another group of preferred mature alpha-amylases are the raw starch degrading alpha-amylases. As used herein, a “raw starch degrading alpha-amylase” refers to an enzyme that can directly degrade raw starch granules below the gelatinization temperature of starch.

Examples of raw starch degrading alpha-amylases include the ones disclosed in WO 2005/003311, U.S. Patent Publication no. 2005/0054071, and U.S. Pat. No. 7,326,548. Examples also include those enzymes disclosed in Table 1 to 5 of the examples in U.S. Pat. No. 7,326,548, in U.S. Patent Publication no. 2005/0054071 (Table 3 on page 15), as well as the enzymes disclosed in WO 2004/020499 and WO 2006/06929 and WO 2006/066579, as well as the enzymes disclosed in the sequence listings and description of WO 2006/069290 (Novozymes A/S) or in WO 2013/006756 (Novozymes A/S), both of which are incorporated herein in their entirety.

In one embodiment, the raw starch degrading alpha-amylase is a GH13_1 amylase.

In one embodiment, the raw starch degrading alpha-amylase enzyme has an amino acid sequence at least 70%, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to the raw starch degrading alpha-amylase shown in EP U.S. Pat. No. 2,981,170 (Novozymes A/S) or in SEQ ID NO: 15 or 16 herein.

### Additional Enzymes

In a preferred embodiment of the first aspect, one or more additional enzyme is added to the dough, said additional enzyme may be selected from the group consisting of a alpha amylase, maltogenic amylase, raw-starch degrading alpha amylase, beta amylase, carboxypeptidase, catalase, cellulase, chitinase, cutinase, cyclodextrin aminopeptidase, glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, raw starch degrading alpha amylase, ribonuclease, transglutaminase, and xylanase.

### Enzyme Compositions

The mature thermostable variant glucoamylase of the invention as well as any additional enzyme(s) may be added in any suitable form, such as, e.g., in the form of a liquid, in particular a stabilized liquid, or it may be added as a substantially dry powder or granulate.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added in any suitable manner, such as individual components (separate or sequential addition of the enzymes) or addition of the enzymes together in one step or one composition.

Granulates and agglomerated powders may be prepared by conventional methods, e.g., by spraying the enzymes onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

### EXAMPLES

### Example 1: Construction of PoAMG Libraries

PoAMG libraries were constructed as follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing JPO-0001 gene) by the following conditions. The resultant PCR fragments were purified by QIAquick Gel extraction kit [QIAGEN], and then introduced into Escherichia coli ECOS Competent E. coli DH5a [NIPPON GENE CO., LTD.]. The plasmid DNAs were extracted from E. coli transformants by MagExtractor plasmid extraction kit [TOYOBO], and then introduced into A. niger competent cells.

### PCR Reaction Mix:

PrimeSTAR Max DNA polymerase [TaKaRa]
Total 25 μl
1,0 μl Template DNA (1 ng/μl)
9,5 μl H₂O
12,5 μl 2× PrimeSTAR Max pre-mix
1,0 μl Forward primer (5 μM)
1,0 μl Reverse primer (5 μM)

### PCR Program:

98 °C/2 min
25× (98 °C/10 sec, 60 °C/15 sec, 72 °C/2 min)
10 °C/hold

### Example 2: Screening for Better Thermostability

B. subtilis libraries constructed as in EXAMPLE 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5N NaOH, 10 ml/L COVE salt, 10 ml/L 1M acetamide), 32 °C for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70 °C for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| Name | |
|---|---|
| | Relative activity of 80 °C/75 °C (%) |
| anPAV498 | 17% |
| JPO-004 | 32% |
| JPO-005 | 15% |
| JPO-006 | 16% |
| JPO-007 | 3% |
| AnPav498 | 13% |
| JPO-009 | 16% |
| JPO-011 | 15% |
| JPO-012 | 15% |
| JPO-013 | 17% |
| JPO-020 | 20% |
| | Relative activity of 80 °C/70 °C (%) |
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |
| | Relative activity of 79 °C/70 °C (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |
| | Relative activity of 77 °C/70 °C (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |
| | Relative activity of 79 °C/77 °C (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| Name | |
|---|---|
| | Relative activity of 77 °C/70 °C (%) |
| JPO-022 | 60% |
| JPO-027 | 67% |
| JPO-042 | 8% |
| JPO-044 | 86% |
| JPO-045 | 67% |
| JPO-046 | 48% |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |
| | Relative activity of 79 °C/77 °C (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
Lists of the relative activity of PoAMG variants when compared
with their parent JPO-063 at different temperatures

| Name | |
|---|---|
| | Relative activity of 79 °C/77 °C (%) |
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |
| | Relative activity of 84 °C/80 °C (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |
| | Relative activity of 82 °C/70 °C (%) |
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |
| | Relative activity of 83 °C/80 °C (%) |
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| Name | |
|---|---|
| | Relative activity of 83 °C/70 °C (%) |
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |
| | Relative activity of 83 °C/80 °C (%) |
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| Name | Relative activity of 84 °C/80 °C (%) |
|---|---|
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| Name | Relative activity of 84 °C/75 °C (%) |
|---|---|
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105LA132R D445N V447S Y504T D566T Q594R F595S |
| JPO-064 | R1A K34Y S103N S105L D445N V447S D566T Q594R F595S |
| JPO-065 | R1A K34Y S103N S105LA132R D445N V447S E501V Y504T D566T Q594R R1A |
| | F595S |
| JPO-066 | R1A K34Y S103N A132R D445N V447S Y504T D566T Q594R F595S |
| JPO-069 | R1A K34Y S103N S105LA132R D445N V447S Y504T D566T V592T |
| JPO-071 | R1A G6S G7T K34Y S103N S105LA132R D445N V447S Y504T D566T Q594R |
| | R1A F595S |
| JPO-074 | R1A K34Y S103N P107LA132R D445N V447S Y504T D566T Q594R F595S |
| JPO-083 | R1A G6S G7T K34Y S103N P107LA132R D445N V447S Y504T D566T Q594R |
| | R1A F595S |
| JPO-084 | R1A G6S G7T K34Y S103N P107LA132R D445N V447S Y504T D566T V592T |
| | R1A Q594R F595S |
| JPO-091 | R1A G6S R7T K34Y S103N P107LA132P D445N V447S Y504T D566T Q594R |
| | F595S |
| JPO-092 | R1A G6S G7T K34Y S103N P107LA132R D445N V447S Y504T D566T T568V |
| | Q594R F595S |
| JPO-093 | R1A G6S G7T K34Y S103N P107LA132P D445N V447S Y504T D566T T568V |
| | Q594R F595S |
| JPO-094 | R1A G6S G7T K34Y S103N P107LA132R D445N V447S S481P Y504T D566T |
| | Q594R F595S |
| JPO-095 | R1A G6S G7T K34Y S103N P107LA132R D445N V447S S481P Y504T D566T |
| | T568V Q594R F595S |
| JPO-096 | R1A G6S G7T K34Y S103N P107LA132P D445N V447S D566T T568V Q594R |
| | F595S |
| JPO-097 | R1A G6S G7T K34Y S103N P107L T110WA132P D445N V447S Y504T D566T |
| | T568V Q594R F595S |
| JPO-098 | R1A G6S G7T K34Y E50R S103N P107LA132P D445N V447S Y504T D566T |
| | T568V Q594R F595S |
| JPO-105 | R1A G6S G7T K34Y S103N P107LA132P D445N V447S E501V Y504T |
| JPO-106 | R1A G6S G7T R31F K34Y S103N P107LA132P D445N V447S Y504T D566T |
| | T568V Q594R F595S |
| JPO-108 | R1A G6S G7T R31F K34Y S103N P107LA132P D445N V447S S481P Y504T |
| | D566T T568V Q594R F595S |
| JPO-109 | R1A G6S G7T K34Y E50R S103N P107LA132P D445N V447S S481P Y504T |
| | D566T T568V Q594R F595S |
| JPO-111 | R1A G6S G7T R31F K34Y S103N P107LA132P D445N V447S S481P E501V |
| | Y504T D566T T568V Q594R F595S |
| JPO-112 | R1A G6S G7T R31F K34Y S103N P107LA132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107LA132P D445N |
| | V447S S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### Example 3: Fermentation of the Aspergillus niger

Aspergillus niger strains were fermented on a rotary shaking table in 500 ml baffled flasks containing 100 ml MU1 with 4 ml 50% urea at 220 rpm, 30 °C. The culture broth was centrifuged (10,000×g, 20 min) and the supernatant was carefully decanted from the precipitates.

### Example 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### Example 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02 °C/sec
Scan range: 37-96 °C.
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### Example 6: PoAMG Activity Assay

Maltodextrin (DE11) assay by GOD-POD method

### Substrate Solution

30 g maltodextrin (pindex #2 from MATSUTANI chemical industry Co., Ltd.)
100 ml 120 mM sodium acetate buffer, pH 5.0
Glucose CII test kit (Wako Pure Chemical Industries, Ltd.)

Twenty ul of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl pH 8.0 to stop reaction. Ten ul of the solution was mixed with 200 ul of the working solution of the test kit then stand at room temperature for 15 min. The absorbance at A505 was read. The activities are listed in Table 3 as relative activity of the PoAMG variant denoted anPAV498.

TABLE 3

| | Td improvement [°C] | Activity at 91 °C |
|---|---|---|
| Variant | (pH 5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: JPO172 in Pulse Bread

Breads were baked in a straight dough baking process with a recipe according to Table 4. Different treatments were made according to Table 5 The bread was baked in open pans. The ingredients were mixed in a spiral mixer into a dough for 3 min at 17 rpm and 7 min at 35 rpm. The doughs were allowed to rest for 10 minutes and divided into 320 g dough pieces. The dough pieces were rounded sheeted and place in baking tins. The tins with the doughs were proofed for 60 min at 32 °C and 86% relative humidity. The proofed doughs were baked in a deck oven for 20 min at 180 °C. 10

The bread was packed 2 hours after baking in sealed plastic bags and stored at room temperature until analysis.

TABLE 4
Recipe

| | Bakers % |
|---|---|
| Flour (Kolibri, Meneba, NL) | 75 |
| Chickpea flour | 25 |
| Water | Variable |
| Yeast | 4 |
| Sucrose | 1.5 |
| Salt | 1.5 |
| Ascorbic acid | 0.01 |
| Calcium Propionate | 0.3 |
| Vital Wheat Gluten | Variable |
| Fungamyl ® G | 10 ppm |

TABLE 5
Treatments

| | 1 | 2 | 3 | 4 | 5 | 6 | 7 | 8 |
|---|---|---|---|---|---|---|---|---|
| Vital | | | | | | 6 | 6 | 6 |
| Wheat | | | | | | | | |
| Gluten, | | | | | | | | |
| % | | | | | | | | |
| Water, | 55 | 55 | 55 | 55 | 55 | 64 | 64 | 64 |
| % | | | | | | | | |
| JPO172, | | 33 | | 33 | | 33 | | 33 |
| mgEP/kg | | | | | | | | |
| flour | | | | | | | | |
| Lipopan | | | 40 | 40 | 40 | | 40 | 40 |
| Xtra, | | | | | | | | |
| ppm | | | | | | | | |
| Novamyl | | | 100 | | | | 100 | |
| G, ppm | | | | | | | | |

The texture of the bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 40% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 40% strain (corresponding to force at time=40s for a bread slice of 25 mm thickness) divided by the force needed to press the probe 10 mm into the crumb (corresponding to force at time=10 s for a bread slice of 25 mm thickness) times 100.

The result form the texture analysis can be found in Table 6 (firmness) and Table 7 (elasticity). The bread with 33 mgEP/kg flour of JPO172 and 40 ppm Lipopan Xtra was more elastic than 100 ppm Novamyl G and 40 ppm Lipopan Xtra. At the same time the breads had equal Firmness.

When additional vital wheat gluten was added to the two treatments the bread with 33 mgEP/kg flour, 40 ppm Lipopan Xtra and 6% vital wheat gluten was more elastic over the whole and had the same firmness during the whole time period studied compared to 100 ppm Novamyl G, 40 ppm Lipopan Xtra and 6% vital wheat gluten.

The addition of 33 mgEP/kg flour of JPO172 alone was also able to improve the elasticity. However, the bread was much harder than the combination of mgEP/kg flour of JPO172 and 40 ppm Lipopan Xtra.

Conclusion: The combination of JPO172 and Lipopan Xtra were able to produce a bread with a combination of low firmness and high elasticity over the whole time period studied.

TABLE 6
Firmness of bread crumb at different timepoints. The letter
behind the number represents significance levels using
a student t-test with a significance level of 0.05.

| Treatment | Day 1 | | Day 7 | | Day 14 | |
|---|---|---|---|---|---|---|
| Control | 633 | A | 1006 | A | 1380 | A |
| 40 ppm Lipopan Xtra | 434 | C | 648 | B | 904 | B |
| 33 mgEP/kg JPO172 | 547 | B | 608 | B | 744 | C |
| 100 ppm Novamyl G + 40 ppm | 298 | D | 400 | CD | 582 | D |
| Lipopan Xtra | | | | | | |
| 33 mgEP/kg JPO172 + 40 ppm | 318 | D | 444 | C | 573 | D |
| Lipopan Xtra | | | | | | |
| 33 mgEP/kg JPO172 + 6% gluten | 308 | D | 356 | DE | 475 | E |
| 33 mgEP/kg JPO172 + 40 ppm | 220 | E | 310 | EF | 360 | F |
| Lipopan Xtra + 6% gluten | | | | | | |
| 100 ppm Novamyl G + 40 ppm | 227 | E | 300 | F | 392 | EF |
| Lipopan Xtra + 6% gluten | | | | | | |

TABLE 7
Elasticity of bread crumb at different timepoints. The letter
behind the number represents significance levels using
a student t-test with a significance level of 0.05.

| Treatment | Day 1 | | Day 7 | | Day 14 | |
|---|---|---|---|---|---|---|
| Control | 59.5 | F | 51.0 | G | 49.5 | F |
| 40 ppm Lipopan Xtra | 59.2 | F | 54.2 | F | 51.1 | E |
| 33 mgEP/kg JPO172 | 64.3 | B | 62.1 | B | 61.9 | BC |
| 100 ppm Novamyl G + 40 ppm | 61.7 | E | 58.7 | E | 59.1 | D |
| Lipopan Xtra | | | | | | |
| 33 mgEP/kg JPO172 + 40 ppm | 63.5 | C | 59.6 | D | 59.8 | D |
| Lipopan Xtra | | | | | | |
| 33 mgEP/kg JPO172 + 6% gluten | 65.6 | A | 64.3 | A | 65.1 | A |
| 33 mgEP/kg JPO172 + 40 ppm | 64.1 | B | 61.8 | B | 62.5 | B |
| Lipopan Xtra + 6% gluten | | | | | | |
| 100 ppm Novamyl G + 40 ppm | 62.7 | D | 60.4 | C | 61.3 | C |
| Lipopan Xtra + 6% gluten | | | | | | |

1. A method of producing a baked or partially baked product comprising:
a) providing a dough comprising added pulse and/or legume protein and a mature thermostable variant of a parent glucoamylase at least 70% identical to SEQ ID NO:1, SEQ ID NO:6, SEQ ID NO:7, SEQ ID NO:8 or SEQ ID NO: 10; and
b) baking or partially baking the dough to produce the baked or partially baked product.

2. The method according to claim 1, wherein the parent glucoamylase is from a species of Penicillium, preferably from Penicillium oxicalum, Penicillium miczynskii, Penicillium russellii or Penicillium glabrum.

3. The method according to claim 1, wherein the mature variant comprises at least one amino acid modification in one or more or all of the positions corresponding to positions 1, 2, 4, 6, 7, 11, 31, 34, 50, 65, 79, 103, 132, 327, 445, 447, 481, 484, 501, 539, 566, 568, 594 and 595 in SEQ ID NO:1.

4. The method according to claim 3, wherein the at least one amino acid modification comprises a substitution in one or more or all of the positions corresponding to positions 1, 2, 4, 11, 65, 79 and 327 in SEQ ID NO: 1.

5. The method according to claim 3, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1.

6. The method according to claim 3, wherein the at least one amino acid modification comprises a substitution in one or more or all of the positions corresponding to positions 1, 6, 7, 31, 34, 50, 103, 132, 445, 447, 481, 501, 539, 566, 568, 594 and 595 in SEQ ID NO: 1.

7. (canceled)

8. The method according to claim 1, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO: 1.

9. The method according to claim 1, wherein the mature thermostable variant has a thermostability improvement (Td) over its parent of at least 5 °C, at least 6 °C, at least 7 °C, or at least 8 °C.

10. The method according to claim 1, wherein the mature thermostable variant has a relative activity at 91 °C of at least 150, at least 200, at least 250, or at least 300 compared to its parent.

11. The method according to claim 1, wherein at least 2% (w/w) of the total flour content is added pulse and/or legume protein, at least 4% (w/w) of the total flour content is added pulse and/or legume protein, at least 6% (w/w) of the total flour content is added pulse and/or legume protein, at least 8% (w/w) of the total flour content is added pulse and/or legume protein, at least 10% (w/w) of the total flour content is added pulse and/or legume protein, or at least 12% (w/w) of the total flour content is added pulse and/or legume protein.

12. The method according to claim 1, wherein the dough also comprises gluten.

13. The method according to claim 1, wherein the dough also comprises at least one added lipase enzyme, or at least one added lipase enzyme comprises a lipase and/or a phospholipase.

14. The method according to claim 13, wherein the at least one added lipase enzyme is added in an amount in the range of 0 to 100 mg enzyme protein/kg flour; in the range of 0 to 50 mg enzyme protein/kg flour; in the range of 0 to 25 mg enzyme protein/kg flour; in the range of 0 to 10 mg enzyme protein/kg flour; in the range of 0 to 5 mg enzyme protein/kg flour; and in the range of 0 to 2.5 mg enzyme protein/kg flour.

15. The method according to claim 1, wherein the dough is mixed:
a) at least 5 minutes at a slow mixing speed in the range of 5-50 rpm, or in the range of 10-40 rpm; and optionally
b) the dough is subsequently mixed at a faster speed.

16. The method according to claim 1, comprising also adding one or more additional enzyme, said additional enzyme selected from the group consisting of a alpha amylase, maltogenic amylase, raw-starch degrading alpha amylase, beta amylase, aminopeptidase, carboxypeptidase, catalase, cellulase, chitinase, cutinase, cyclodextrin glycosyltransferase, deoxyribonuclease, esterase, glucan 1,4-alpha-maltotetrahydrolase, glucanase, beta glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, pullulanase, ribonuclease, transglutaminase, and xylanase.

17. The method according to claim 4, wherein the at least one amino acid modification comprises a substitution in one or more or all of the positions corresponding to R1A, P2N, P4S, P11F, T65A, K79V and Q327F in SEQ ID NO: 1.

18. The method according to claim 5, wherein the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO: 1.

19. The method according to claim 6, wherein the at least one amino acid modification comprises a substitution in one or more or all of the positions corresponding to R1A, G6S, G7T, R31F, K34Y, E50R, S103N, A132P, D445N, V447S, S481P, E501A, N539P, D566T, T568V, Q594R and F595S in SEQ ID NO: 1.

20. The method according to claim 8, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO: 1.

21. The method according to claim 13, where the at least one added lipase enzyme comprises a mature lipase having an amino acid sequence at least 70% identical to one or more of the sequences of SEQ ID NO: 17, SEQ ID NO: 18, SEQ ID NO: 19, SEQ ID NO: 20 and SEQ ID NO: 21.


SEQUENCE LISTING (16 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, synthetic construct
  REGION 1..595: note = AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, synthetic construct
  REGION 1..595: note = AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, synthetic construct
  REGION 1..595: note = AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, synthetic construct
  REGION 1..595: note = AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, synthetic construct
  REGION 1..595: note = AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 11 - protein, 686 aa, Bacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 12 - protein, 530 aa, Pseudomonas saccharophila
DQAGKSPAGVRYHGGDEIILQGFHWNVVREAPNDWYNILRQQASTLAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 13 - protein, 429 aa, Pseudomonas saccharophila
DQAGKSPAGVRYHGGDEIILQGFHWNVVREAPYNWYNILRQKASTIAADGFSAIWMPVPWRDFSSWTDGDKSGGGEGYFWHDFNKNGLYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDLWMSDSADSSFCVGELWKKPSEYPPWDWRNRASWQEIIKDWSDRAKCPVFDFALKERMQNGSVADWKHGLNGNPDPRWREVAVTFVDNHDTGYSPGQNGGQHKWPLPDGLIRQAYAYILTSPGTPVVYWPHMYDWGYGDFIRQLIQVRRTAGVRADSAISFHSGYSGLVATVSGSQQTLVVALNSDLDNPGQVASGSFSEAVNAENGQVRVWRSGSGDGGGNDGG

SEQ ID NO: 14 - protein, 429 aa, Pseudomonas saccharophila
DQAGKSPAGVRYHGGDEIILQGFHWNVVREAPYNWYNILRQQASTIAADGFSAIWMPVPWRDFSSWTDGDKSGGGEGYFWHDFNKNGRYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDSWMSDSADSSFCVGELWKEPSEYPPWDWRNTASWQQIIKDWSDRAKCPVFDFALKERMQNGSVADWKHGLNGNPDPRWREVAVTFVDNHDTGYSPGQNGGQHKWPLQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 15 - protein, 449 aa, synthetic construct
PLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSA

SEQ ID NO: 16 - protein, 583 aa, synthetic construct
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR